(19) United States
(12) Patent Application Publication
(10) Pub. No.: US 2010/0167801 A1
(43) Pub. Date: Jul. 1, 2010

(54) KIDS PERSONAL HEALTH RECORDS FED INTO VIDEO GAMES

(75) Inventors: Chris Demetrios Karkanias, Sammamish, WA (US); Kim Cameron, Bellevue, WA (US); Vladimir Sadovsky, Bellevue, WA (US); Hong Choing, Collegeville, PA (US); Mary P. Czerwinski, Woodinville, WA (US)

Correspondence Address: Kayla D. Brant;Lee & Hayes, PLLC, 601 W. Riverside Avenue, Suite 1400, Spokane, WA, 99201, US

(73) Assignee: MICROSOFT CORPORATION, Redmond, WA (US)

(21) Appl. No.: 12/346,949

(22) Filed: Dec. 31, 2008

(51) Int. Cl. A63F 9/24; A61B 5/00; G06Q 50/00

(52) U.S. Cl. 463/7; 600/301; 705/3

(57) ABSTRACT

The claimed matter provides systems and/or techniques that regulate and/or prescribe an individual's behavior while playing electronic games. The system includes mechanisms and/or modalities that identify physical and/or mental activities similar to those undertaken by a game character and that are appropriate to the fitness or mental capabilities of the individual. It requests the individual to perform the activities selected during the execution of the electronic game, monitors the individual's performance of the activity, and reproduces and associates the individual's actions in performing the selected task to the game character during execution of the electronic game. Further, it enhances or diminishes attributes of the game character based on the intensity of the individual's performance of the selected activity.

### BACKGROUND

The incidence of childhood obesity is on the rise and has reached epidemic proportions. In recent decades the number of obese children, regardless of age, race, or gender, has doubled in the United States. As children spend more time in front of televisions, computers, video screens, and/or playing electronic games, their physical activity levels have decreased commensurately.

Obese children and adolescents have shown an alarming increase in the incidence of type 2 diabetes, also known as adult-onset diabetes, many of these children have high cholesterol and elevated blood pressure levels which are risk factors for heart disease. Obese children can suffer from sleep apnea (e.g., interrupted breathing while sleeping) which can lead to problems with learning and memory. Obese children have a higher incidence of other ailments, such as, orthopedic problems, liver disease, and asthma, for example. Moreover, overweight adolescents have a more than 70 percent chance of becoming overweight or obese adults.

To date, the most positive impact that electronic games are said to have had on children is that playing such games may improve a player's manual dexterity and computer literacy. Ever improving technology has also provided players with better graphics that give a more realistic virtual experience. Major studies indicate that children on average spend just over an hour playing electronic games, and boys typically spend substantially more time playing electronic games than do girls, regardless of age. On any given day, 44% of boys report playing electronic games compared to 17% of girls. This quality and pervasiveness thus makes the electronic game industry a very powerful force in many children's lives. Moreover, with the vast repositories of data available for access and data mining on the Internet, especially with regard to the medical aspects of human conditioning (e.g., exercise regimens, dietary requirements, and the like), such information can be gainfully utilized for the beneficial purposes of behavior modification in children.

The subject matter as claimed is directed toward resolving or at the very least mitigating, one or all the issues elucidated above.

### SUMMARY

The following presents a simplified summary in order to provide a basic understanding of some aspects of the disclosed subject matter. This summary is not an extensive overview, and it is not intended to identify key/critical elements or to delineate the scope thereof Its sole purpose is to present some concepts in a simplified form as a prelude to the more detailed description that is presented later. A system that regulates behavior, comprising:

The claimed subject matter in accordance with an aspect provides systems and/or techniques that regulate user behavior while utilizing or playing electronic games. The mechanisms and/or modalities set forth herein provide access to personal health records associated with an individual, utilization of electronic game character attributes associated with a game persona selected by the individual to play the electronic game, information gleaned from the personal health records, and/or data received or acquired from sensors affiliated with and/or dispersed around the individual to identify likely activities that the game persona can request the individual to perform during game play. The devices and/or processes utilized by the matter claimed and disclosed here can thereafter monitor the sensors associated with, and/or dispersed around, the individual to gauge whether or not the individual has commenced, or completed performance of, the activities requested of the individual. Based at least in part on the intensity of the performance of the activities requested of the individual or information from the personal health records associated with the individual, the devices and/or processes disclosed and/or claimed herein can augment or reduce attributes associated with the game character.

To the accomplishment of the foregoing and related ends, certain illustrative aspects of the disclosed and claimed subject matter are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a machine-implemented system that regulates or exerts influence over behavior in accordance with the claimed subject matter.

FIG. 2 provides a more detailed depiction of an illustrative monitor component that regulates user behavior in accordance with an aspect of the claimed subject matter.

FIG. 3 provides a more detailed depiction of an illustrative entertainment platform that influences user behavior in accordance with an aspect of the claimed subject matter.

FIG. 4 illustrates a system implemented on a machine that regulates user behavior in accordance with an aspect of the claimed subject matter.

FIG. 5 provides a further depiction of a machine implemented system that regulates electronic game user's behavior in accordance with an aspect of the subject matter as claimed.

FIG. 6 illustrates yet another aspect of the machine implemented system that regulates user behavior in accordance with an aspect of the claimed subject matter.

FIG. 7 depicts a further illustrative aspect of the machine implemented system that regulates a user's behavior in accordance with an aspect of the claimed subject matter.

FIG. 8 illustrates another illustrative aspect of a system implemented on a machine that regulates a user's behavior in accordance of yet another aspect of the claimed subject matter.

FIG. 9 illustrates another illustrative aspect of a system implemented on a machine that regulates a user's behavior in accordance of yet another aspect of the claimed subject matter.

FIG. 10 illustrates a flow diagram of a machine implemented methodology that influences a user's behavior in accordance with an aspect of the claimed subject matter.

FIG. 11 depicts a flow diagram of a method implement on a machine that regulates aspects of a user's behavior in accordance with an aspect of the claimed subject matter.

FIG. 12 illustrates a block diagram of a computer operable to execute the disclosed system in accordance with an aspect of the claimed subject matter.

FIG. 13 illustrates a schematic block diagram of an illustrative computing environment for processing the disclosed architecture in accordance with another aspect.

### DETAILED DESCRIPTION

The subject matter as claimed is now described with reference to the drawings, wherein like reference numerals are used to refer to like elements throughout. In the following description, for purposes of explanation, numerous specific details are set forth in order to provide a thorough understanding thereof. It may be evident, however, that the claimed subject matter can be practiced without these specific details. In other instances, well-known structures and devices are shown in block diagram form in order to facilitate a description thereof.

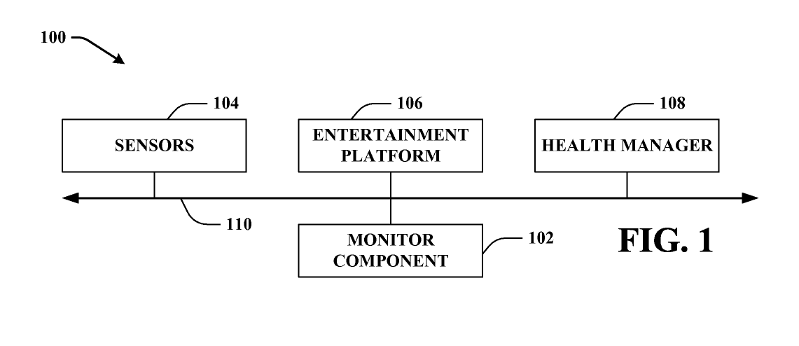

FIG. 1 illustrates a machine implemented system 100 that influences or prescribes user behavior in accordance with an aspect of the claimed subject matter. System 100 can include monitor component 102 that accesses or acquires data and/or information from a multitude of sources including sensors 104, entertainment platform 106, and/or health manager 108 via network topology and/or cloud 110. Monitor component 102 can utilize received or obtained information to control or regulate aspects of a users' behavior. Additionally and/or alternatively, monitor component 102 can employ accessed or acquired data to dynamically influence characteristics of an electronic game, or attributes of electronic game characters or electronic game persona (e.g., attributes that can include traits such as physical strength, manual dexterity, mental acuity, idiosyncratic tendencies, mythical or superhuman capabilities, skills with respect to particular physical, mental, or sensory activities, and the like).

As illustrated, monitor component 102 can be in continuous and/or operative, or intermittent but sporadic communication with sensors 104, entertainment platform 106, and/or health manager 108 via network topology and/or cloud 110. Monitor component 102 can be implemented entirely in hardware and/or a combination of hardware and/or software in execution. Further, monitor component 102 can be incorporated within and/or associated with other compatible components. Moreover, monitor component 102 can be any type of machine that includes a processor and/or is capable of effective communication with network topology and/or cloud 110. Illustrative machines that can comprise monitor component 102 can include cell phones, smart phones, laptop computers, notebook computers, Tablet PCs, consumer and/or industrial devices and/or appliances, hand-held devices, personal digital assistants, server class machines and/or computing devices and/or databases, multimedia Internet enabled mobile phones, multimedia players, automotive components, avionics components, and the like.

Sensors 104 can be any mechanism or device that can be utilized to measure or observe activity or inactivity associated with a particular individual. Sensors 104 can include, without limitation, microphones, cameras, pedometers, accelerometers, heart rate monitors, thermometers, devices associated with exercise machines such as elliptical machines, treadmills, exercise bicycles, step machines, devices incorporated into running tracks, swimming pools, basketball courts, and the like, devices utilized in home and/or office environments to monitor ambient variables (e.g., thermostats, motion detectors, and the like), or any other device or component that can be utilized to monitor activity or inactivity associated with users of system 100. Further, sensors 104 can be included with, or incorporated in, textiles, fabrics, clothing, jewelry, or any item that can be worn. Additionally, sensors 104 can be Radio Frequency Identification (RFID) devices, or can incorporate or subsume Radio Frequency Identification (RFID) devices. For example, sensors 104 can be a wrist band that has embedded therein a Radio Frequency Identification (RFID) tag or chip that can continuously obtain information (e.g., blood pressure, body temperature, perspiration, pupil dilation, heart rate, location information from global positioning systems, . . . ) from the wearer of the wrist band. Additionally, sensors 104 can be implemented entirely in hardware and/or as a combination of hardware and/or software in execution. Further, sensors 104 can be any type of mechanism, machine, device, facility, and/or instrument that includes a processor and is capable of effective and/or operative communication with network topology and/or cloud 110. Illustrative mechanisms, machines, devices, facilities, and/or instruments that can comprise sensors 104 can include Tablet PCs, server class computing machines, laptop computers, notebook computers, desktop computers, cell phones, smart phones, consumer appliances and/or instrumentation, industrial devices and/or components, hand-held devices, personal digital assistants, multimedia Internet enabled phones, Global Positioning Systems (GPS), multimedia players, and the like.

Entertainment platform 106 can be any interactive electronic or electrical device that manipulates signals associated with a presentation device (e.g., television, monitor, etc.) to display an electronic game or game characters, persona, or sprites (e.g., Movable Object Blocks (MOB), Blitter Objects (BOB), 3D-sprites, Z-sprites, and the like) associated with the electronic game. Entertainment platform 106 can be implemented entirely in hardware and/or as a combination of hardware and/or software in execution. Further, entertainment platform 106 can be incorporated within and/or associated with other compatible components. Additionally, entertainment platform 106, like monitor component 102 and sensors 104, can be effectuated on any type of mechanism, machine, device, facility, and/or instrument that includes a processor and is capable of effective and/or operative communication with network topology and/or cloud 110. Illustrative mechanisms, machines, devices, facilities, and/or instruments of production that can comprise entertainment platform 106 can include Tablet PCs, server class computing machines and/or databases, laptop computers, notebook computers, desktop computers, cell phones, smart phones, consumer appliances and/or instrumentation, industrial devices and/or components, hand-held devices, personal digital assistants, gaming consoles, multimedia Internet enabled phones, multimedia players, etc.

Health manager 108 can be an online repository and/or directed search facility that persists or stores an individual's health data ranging from test results to physician's reports to daily measurements of weight or blood pressure. Individuals can then have access to their records at any time, anywhere, via network topology and/or cloud 110. Affiliated medical practitioners, medical offices, and/or hospitals can, for instance, easily forward test results in digital form to health manager 108, and individuals (e.g. patients) can in turn authorize selected medical practitioners, medical offices, hospitals, components owned or controlled by the individual (e.g., monitor component 102, entertainment platform 106, . . . ), and the like, to access various carefully circumscribed aspects of their personal data. Moreover, health manager 108 can provide centralized storage and access to personal health records so that users can easily access their personal records from anywhere and at anytime regardless of the access platform (e.g., cell phone, smart phone, gaming console, and the like). Further, entertainment platform 106 can access health manger 108 based at least in part on a unique user identity wherein the user identity can be associated with a network identity or online gaming persona, or the user identity can be acquired through a token persisted on portable flash devices (e.g., card or Universal Serial Bus (USB) flash devices). Additionally and/or alternatively, health manager 108 can also provide directed and/or targeted vertical search capabilities that can provide more relevant results than generalist search engines. For instance, a search actuated on health manager 108 can allow individuals to specifically tailor their search queries based on their persisted health records, past queries, and the like, and can receive in return results that are most relevant to each individual's situation. In addition, an offline portable record store can act as health manager 108, wherein entertainment platform 106 can call into functions exposed an active portable record store (e.g., when the portable record store is online). For instance, a cell phone can have persisted personal records and can respond to service calls (e.g., via Bluetooth) to provide or extract records from associated flash memory.

Health manager 108, like monitor component 102, sensors 104, and/or entertainment platform 106, can be implemented entirely in hardware and/or as a combination of hardware and/or software in execution. Further, health manager 108 can be any type of engine, machine, instrument of conversion, or mode of production that includes a processor and/or is capable of effective and/or operative communications with network topology and/or cloud 110. Illustrative instruments of conversion, modes of production, engines, mechanisms, devices, and/or machinery that can comprise and/or embody health manager 108 can include desktop computers, server class computing devices and/or databases, cell phones, smart phones, laptop computers, notebook computers, Tablet PCs, consumer and/or industrial devices and/or appliances and/or processes, hand-held devices, personal digital assistants, multimedia Internet enabled mobile phones, multimedia players, offline portable record stores, and the like.

Network topology and/or cloud 110 can include any viable communication and/or broadcast technology, for example, wired and/or wireless modalities and/or technologies can be utilized to effectuate the claimed subject matter. Moreover, network topology and/or cloud 110 can include utilization of Personal Area Networks (PANs), Local Area Networks (LANs), Campus Area Networks (CANs), Metropolitan Area Networks (MANs), extranets, intranets, the Internet, Wide Area Networks (WANs)—both centralized and/or distributed—and/or any combination, permutation, and/or aggregation thereof. Additionally, network topology and/or cloud 110 can include or encompass communications or interchange utilizing Near-Field Communications (NFC) and/or communications utilizing electrical conductance of the human skin, for example.

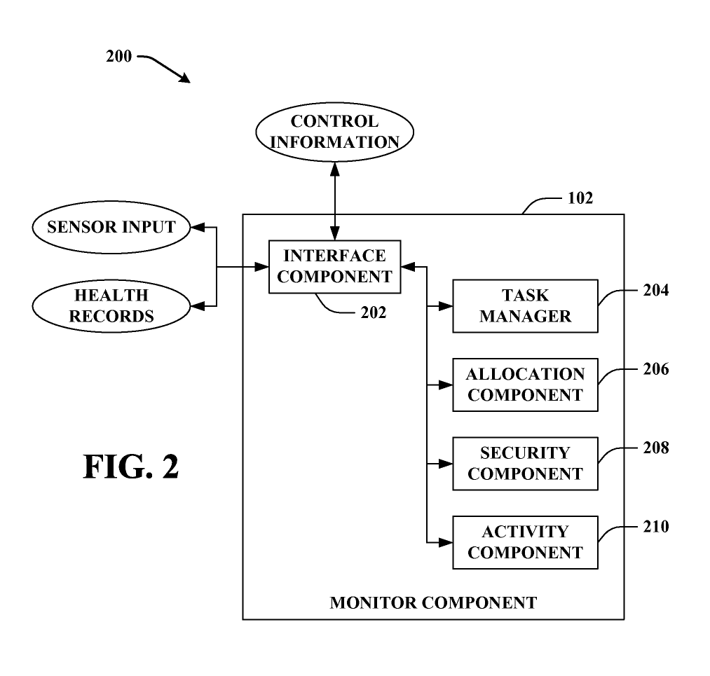

FIG. 2 provides a more detailed depiction 200 of monitor component 102 in accordance with an aspect of the claimed matter that can actively and/or passively acquire or access input, such as, for example, input from sensors 104, information associated with electronic games or game characters or game personas, and/or health related records from health manager 108 via interface component 202. Interface component 202 (hereinafter referred to as “interface 202”) can receive and/or disseminate, communicate, and/or partake in data interchange with a plurality of disparate sources and/or components. For instance, interface 202 can receive and/or transmit data from, or to, a multitude of sources, such as, for example, data associated with health records obtained from health manager 108, activity levels obtained from sensors 104 associated with a game player, and/or control information obtained from entertainment platform 106. Additionally and/or alternatively, interface 202 can obtain and/or receive data associated with usernames and/or passwords, sets of encryption and/or decryption keys, client applications, services, users, clients, devices, and/or entities involved with a particular transaction, portions of transactions, and thereafter can convey the received or otherwise acquired information to one or more of task manager 204, allocation component 206, security component 208, and/or activity component 210, for subsequent utilization, processing, and/or analysis. To facilitate its objectives, interface 202 can provide various adapters, connectors, channels, communication pathways, etc. to integrate the various components included in system 200, and more particularly, monitor component 102, into virtually any operating system and/or database system and/or with one another. Additionally and/or alternatively, interface 202 can provide various adapters, connectors, channels, communication modalities, and the like, that can provide for interaction with the various components that can comprise system 200, and/or any other component (external and/or internal), data, and the like, associated with system 200.

Monitor component 102 in accordance with an aspect, having acquired or accessed input from various sources can identify activities associated with an electronic game, game character, or game persona chosen or selected by a user or electronic game player. For instance, monitor component 102 can identify appropriate activities that can be performed within the ambit of the electronic game, or by a game character/persona selected by the game player based at least in part on the electronic game type (e.g., action game), health records associated with the game player, and/or information regarding the game player's current or immediately preceding activity levels. The activities identified and/or selected by monitor component 102 can require physical activity (e.g. running for a prescribed amount of time or over a defined distance), mental activity (e.g. performing basic mental arithmetic for a certain duration or within a fixed amount of time), or a combination of physical and mental activity (e.g., orienteering where a physical activity—hiking—is combined with mental activities such as map reading and utilization of a compass). As will be appreciated, the activities identified and associated with a game or game characters can be varied and many, requiring many differing levels of skill, activity, capabilities and/or achievement, however, each activity identified can be specifically selected and individuated for the game player or user of the system. Thus, monitor component 102 can select and identify an extremely rigorous activity that the game requires or the game persona performs, but can nevertheless ameliorate or mitigate the degree of exertion required to accomplish the task by substituting extremely demanding activity with less physically demanding aspects, such mitigation or amelioration of physically or mentally demanding activity by monitor component 102 can be based on one or more characteristics obtained from health records persisted on or associated with health manager 108, and/or information acquired from sensors 104. Additionally, monitor component 102 can dynamically adjust the assigned or prescribed tasks during performance depending on data continuously obtained from sensors 104 and in conformance with indications provided by health records received from health manager 108.

Monitor component 102 can dynamically generate activity tasks that can be or should be performed by the individual playing the game. The activity tasks selected by monitor component 102 can be individuated or individualized based at least in part on age, sex, medical records, current medical conditions, past activities, etc. Monitor component 102 can also ensure that individuals playing the electronic game or employing a game persona or game character associated with the electronic game performs the assigned task. Monitor component 102 can ensure that the individual playing the electronic game or partaking in the electronic game vicariously through a game sprite or game persona performs the set task by continuously monitoring and/or analyzing data received from sensors 104. Additionally, monitor component 102, based at least in part on the amount of activity detected from the individual playing the electronic game and in fulfillment of the task assigned, can provide a reward scheme. Such a reward scheme can include assigning points or credits that the user can utilize in playing the game (e.g., avoiding demanding tasks or receiving tasks of less demanding physical or mental rigor), or monetized credits that can be employed or utilized to purchase other electronic games, gaming paraphernalia, or exercise equipment to be utilized in conjunction with entertainment platform 106, for example. Moreover, monitor component 102 can employ motions associated with the individuals assigned activity to contemporaneously actuate the game character to replicate or mimic the activity or motion in the gaming environment (e.g., if the game user has been asked to scale a wall, the game persona can also scale the wall at the same time that the game user is partaking in this activity; the speed or skill with which the game character accomplishes this task can be attuned to the game player's performance).

In accordance with the foregoing, monitor component 102 can employ task manger 204 that can identify activities that are performed by the game persona or game character that are suitable to be performed by the game player. For instance, task manager 204 can employ health records acquired or requested from health manager 108 in conjunction with game information received from entertainment platform 106 and data obtained from sensors 104 to identify appropriate activities within the capabilities of the game player. The activities selected by task manager 204 can be individuated and/or customize to cater for specific requirements identified in the user's health records. For example, health records associated with the game player and received from health manager 108 may indicate that the game player requires rehabilitation in some physical aspect (e.g. flexing hip joints after surgery). Accordingly, task manager 204 can select activities that specifically focus and concentrate on this aspect.

Monitor component 102 can further include allocation component 206 that can determine durations, distances, or intensity levels over or during which an identified or selected activity should be performed. Such determination as performed by allocation component 206 can be based at least in part on input received from sensors 104, game persona characteristics or attributes obtained from entertainment platform 106, and/or health records received from health manager 108. Accordingly, allocation component 206, based at least in part on inputs received, can intensify or lessen the rigor of the particular activity according to the game player's needs and capabilities.

As will be appreciated, access to, or more specifically unauthorized access to, personal health records can be a highly emotive topic. Thus, in order to prevent unauthorized access to health records robust security features need to be implemented and/or utilized. Accordingly, monitor component 102 can include security component 208 that can utilize cryptographic keys to unlock or lock sensitive data or portions thereof. Security component 208 can selectively protect/secure portions of the health record, granting selective access to individuals or organizations of the users choosing. For instance, an individual may wish that a heart specialist only have access to records related specifically to the individual's heart, or that a neurologist only have access to records involving the individual's brain. In order to facilitate these objectives, security component 208 can employ various encryption schemes that can be based at least in part on asymmetric public key systems (PKS) or symmetric key systems (e.g., block ciphers or stream ciphers). Examples of cryptographic asymmetric public key systems that can be employed by the claimed subject matter include techniques such as Diffie-Hellman, Digital Signature Standard (DSS), various elliptic curve modalities, RSA, Cramer-Shoup, Merkle-Hellman ‘knapsack’ algorithms, Paillier cryptosystems, etc. Examples of symmetric key systems that can be employed with equal utility by the claimed subject matter can include techniques such as Twofish, the Advanced Encryption Standard (AES), Blowfish, Serpent, RC4, Triple Data Encryption Standard (TDES), International Data Encryption Algorithm (IDEA), and the like. Moreover it should be noted, solely for the purposes of exposition rather than limitation, that the key lengths that can be utilized for purposes of encryption or decryption can be greater than or less than the 2⁷- or 2⁸-bits currently and typically practicable in this field of endeavor. Moreover, security component 208 can also utilize biometrics (e.g., retinal scans, iris scans, finger prints, perspiration analysis, and the like) to ensure the security and access of personal health records associated with health manager 108. Further, security component 208 can also use hardware tokens or smart tokens that can provide or supply cryptographic information in order to control access to sensitive information.

Additionally, monitor component 102 can also include activity component 210 that can ensure that individuals playing the electronic game or employing the game persona or game character in association with electronic game perform the task assigned. Activity component 210 can continuously monitor input received from sensors 104 associated with the individual playing the electronic game to ascertain that the individual is complying with the assigned task. Further, activity component 210 can dynamically modify prospective tasks and/or ongoing or tasks currently being performed based at least in part on health records received from health manager 108 and/or data received from sensors 104. For example, activity component 210 can detect that the individual playing or partaking in the activity associated with the electronic game is over exerting him or herself and that his or her heart rate is reaching critical levels, thus activity component 210 can dynamically institute one or more “cool-down” aspects of the activity and in this manner can prevent the individual from sustaining injury.

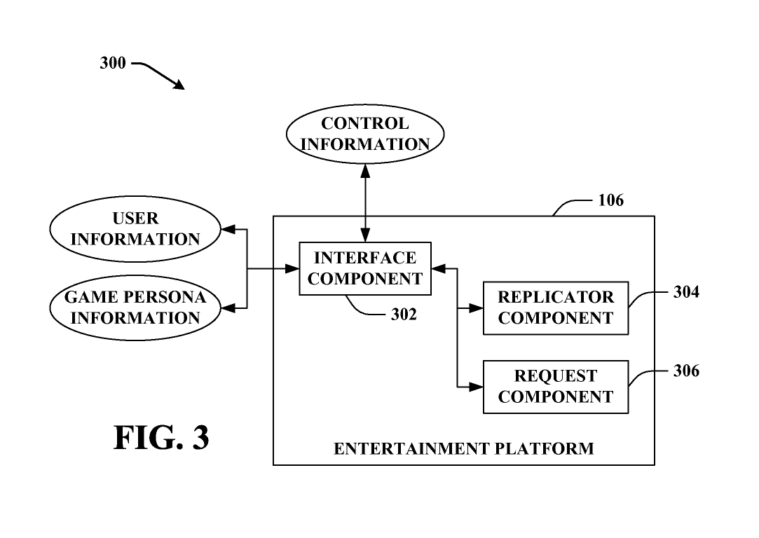

FIG. 3 provides a more detailed depiction 300 of entertainment platform 106 in accordance with an aspect of the claimed subject matter. As illustrated, entertainment platform 106 can include interface component 302, replicator component 304, and request component 306. Because much of the configuration and operation of interface component 302 is substantially similar to that presented and detailed in connection with FIG. 2 and interface component 202, a detailed description of such features has been omitted to avoid needless repetition and for the sake of brevity and conciseness of exposition.

In order to progress or advance in an electronic game, a game character or game persona typically needs to be energized or invigorated. Such invigoration of game characters or game personas can stem from player activity. In accordance with an aspect of the claimed subject matter, player activity can inform the game and game activity can motivate or actuate user activity, albeit in disguise or surreptitiously. As stated earlier, activities can include physical goals, cognitive tasks, a combination of physical and/or cognitive tasks, educational attainments, etc. Nevertheless, the activity that either informs the game or the game activity that motivates individual can be specific to the user playing the game. Consequently game characters or game personas associated with the individual can have skills and attributes that can be enhanced or reduced based on the intensity of performance by the individual playing the game.

Accordingly, replicator component 304 can receive via interface component 302 information associated with a user from sensors 104, health related records from health manager 108, and/or utilize information regarding game persona or game character information (e.g., attributes related to the game persona's skills, mental acuity, typical activities within the game character's or game persona's repertoire, and the like). Based at least in part on the characteristics or attributes ascribed to the game character or game persona, information obtained from health records and/or sensor data, for example, replicator component 304 can identify tasks associated with the game character's or game persona's repertoire of skills, strengths, and/or attributes that the user can successfully accomplish. Replicator component 304 can then utilize the services of request component 306 to direct the user to commence performance of the assigned tasks, or portions of the tasks. For instance, the identified tasks can involve a “mission” (e.g., capture a fortress) wherein a number of subtasks (e.g., swim a moat, scale the outer fortress wall, run across open terrain to a inner sanctum wall, conduct hand-to-hand combat against innumerable assailants/defenders, . . . ) are required for successful completion, such that the user or game player can be solicited by request component 306 to swim an assigned distance in a swimming pool, perform rock climbing at a climbing wall, run 1500 meters in a fixed period of time, . . . , in order to successfully capture the fortress and complete the mission. Contemporaneously with the user undertaking performance of the “mission” the game persona can also perform the mission in the gaming environment wherein the performance of the game user can have a direct correspondence on the performance of the game persona (e.g., the more effort the game player puts into the performance of the tasks, the more successful the game persona or character will be in successfully completing the mission in the gaming environment).

Thus, once request component 306 has initiated the user or game player on the course to tackle a particular task or set of tasks, replicator component 304 can monitor sensors 104 associated with the user or game player for activity or inactivity in the performance of the assigned tasks or set of tasks. Where replicator component 304 detects activity it can replicate or project the detected activity onto the game persona or game character in the gaming environment so that the game character or persona mimics the actions of the game player. Replicator component 304 can also enhance or diminish the game persona's abilities (e.g., strength or speed) where the game player is being indolent or is not performing the tasks in accordance with his or her full potential. Conversely, where replicator component 304 detects that the game player is struggling (e.g., through increased heart rate, elevated blood pressure, profuse sweating, etc.) it can, for example, dynamically change the pattern of play, introduce “cool down” activities, or substitute less demanding activities. Similarly, where replicator component 304 perceives that the activities assigned are below the capabilities of the game player, it can automatically and/or dynamically enhance or augment the activities being requested of the game player with more challenging tasks.

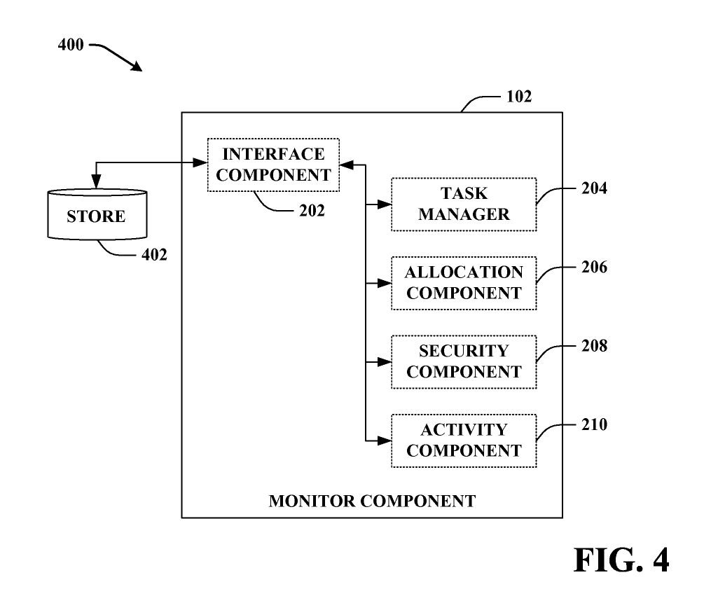

FIG. 4 depicts an aspect of a system 400 that effectuates and facilitated regulation or control of behavior in accordance with an aspect of the claimed subject matter. System 400 can include store 402 that can include any suitable data necessary for monitor component 102 to facilitate its objectives. For instance, store 402 can include information regarding user data, data related to a portion of a transaction, credit information, historic data related to a previous transaction, a portion of data associated with purchasing a good and/or service, a portion of data associated with selling a good and/or service, geographical location, online activity, previous online transactions, activity across disparate networks, activity across a network, credit card verification, membership, duration of membership, communication associated with a network, buddy lists, contacts, questions answered, questions posted, response time for questions, blog data, blog entries, endorsements, items bought, items sold, products on the network, information gleaned from a disparate website, information obtained from the disparate network, ratings from a website, a credit score, geographical location, a donation to charity, or any other information related to software, applications, web conferencing, and/or any suitable data related to transactions, etc.

It is to be appreciated that store 402 can be, for example, volatile memory or non-volatile memory, or can include both volatile and non-volatile memory. By way of illustration, and not limitation, non-volatile memory can include read-only memory (ROM), programmable read only memory (PROM), electrically programmable read only memory (EPROM), electrically erasable programmable read only memory (EEPROM), or flash memory. Volatile memory can include random access memory (RAM), which can act as external cache memory. By way of illustration rather than limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink® DRAM (SLDRAM), Rambus® direct RAM (RDRAM), direct Rambus® dynamic RAM (DRDRAM) and Rambus® dynamic RAM (RDRAM). Store 402 of the subject systems and methods is intended to comprise, without being limited to, these and any other suitable types of memory. In addition, it is to be appreciated that store 402 can be a server, a database, a hard drive, and the like, and further can include removable and/or portable personal memory or storage devices (e.g., flash cards or flash memory) that can be interchangeably transported between aspects and components of the claimed subject matter.

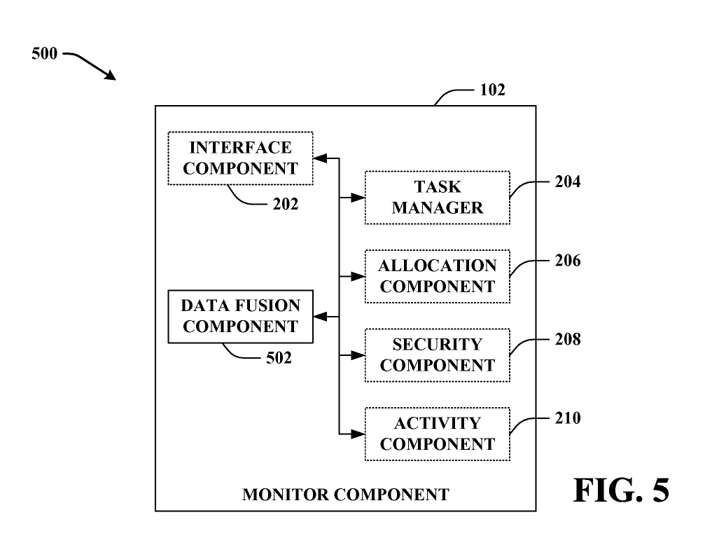

FIG. 5 provides yet a further depiction of a system 500 that facilitates and/or effectuates regulation of a user's physical or mental activity in accordance with an aspect of the claimed subject matter. As depicted, system 500 can include a data fusion component 502 that can be utilized to take advantage of information fission which may be inherent to a process (e.g., receiving and/or deciphering inputs) relating to analyzing inputs through several different sensing modalities. In particular, one or more available inputs may provide a unique window into a physical environment (e.g., an entity inputting instructions) through several different sensing or input modalities. Because complete details of the phenomena to be observed or analyzed may not be contained within a single sensing/input window, there can be information fragmentation which results from this fission process. These information fragments associated with the various sensing devices may include both independent and dependent components.

The independent components may be used to further fill out (or span) an information space; and the dependent components may be employed in combination to improve quality of common information recognizing that all sensor/input data may be subject to error, and/or noise. In this context, data fusion techniques employed by data fusion component 502 may include algorithmic processing of sensor/input data to compensate for inherent fragmentation of information because particular phenomena may not be observed directly using a single sensing/input modality. Thus, data fusion provides a suitable framework to facilitate condensing, combining, evaluating, and/or interpreting available sensed or received information in the context of a particular application.

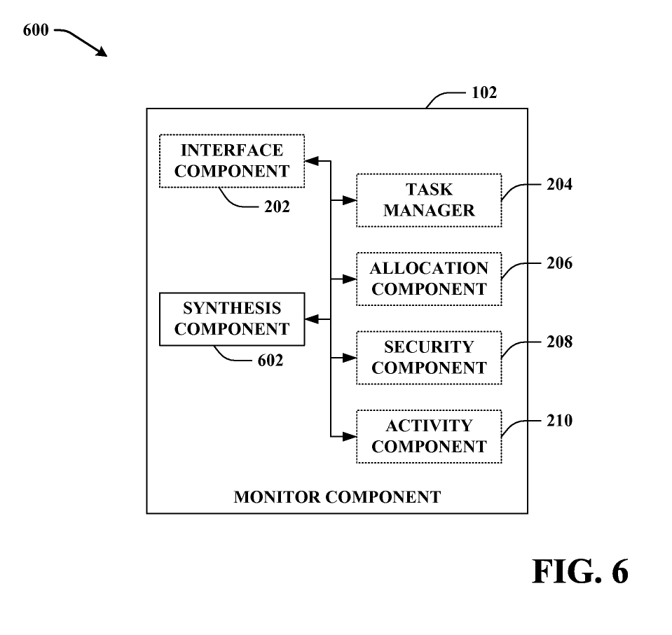

FIG. 6 provides a further depiction of a system 600 that facilitates and/or effectuates control over physical or mental activities undertaken by a user in accordance with an aspect of the claimed subject matter. As illustrated monitor component 102 can, for example, employ synthesis component 602 to combine, or filter information received from a variety of inputs (e.g., text, speech, gaze, environment, audio, images, gestures, noise, temperature, touch, smell, handwriting, pen strokes, analog signals, digital signals, vibration, motion, altitude, location, GPS, wireless, etc.), in raw or parsed (e.g. processed) form. Synthesis component 602 through combining and filtering can provide a set of information that can be more informative, or accurate (e.g., with respect to an entity's communicative or informational goals) and information from just one or two modalities, for example. As discussed in connection with FIG. 5, the data fusion component 502 can be employed to learn correlations between different data types, and the synthesis component 602 can employ such correlations in connection with combining, or filtering the input data.

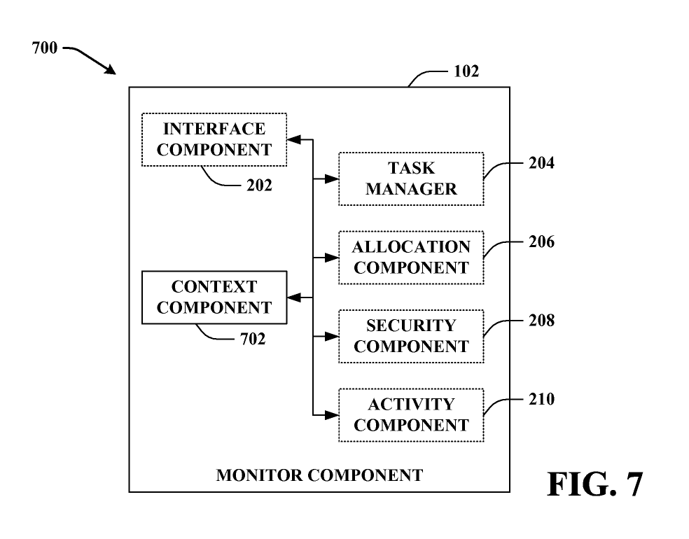

FIG. 7 provides a further illustration of a system 700 that can effectuate and/or facilitate control and/or regulation over an individual's physical or mental activities in accordance with an aspect of the claimed subject matter. As illustrated monitor component 102 can, for example, employ context component 702 to determine context associated with a particular action or set of input data. As can be appreciated, context can play an important role with respect understanding meaning associated with particular sets of input, or intent of an individual or entity. For example, many words or sets of words can have double meanings (e.g., double entendre) or are homonyms (e.g., two, to, too, wind [i.e., noun: air in natural motion], wind [i.e., verb; to roll or coil into a ball, or spool onto a bobbin], . . . ), and without proper context of use or intent of the words the corresponding meaning can be unclear thus leading to increased probability of error in connection with interpretation or translation thereof. The context component 702 can provide current or historical data in connection with inputs to increase proper interpretation of inputs. For example, time of day may be helpful to understanding an input—in the morning, the word “drink” would likely have a high a probability of being associated with coffee, tea, or juice as compared to being associated with a soft drink or alcoholic beverage during later hours. Context can also assist in interpreting uttered words that sound the same (e.g., steak and, and stake). Knowledge that it is near dinnertime of the user as compared to the user camping would greatly help in recognizing the following spoken words “I need a steak/stake”. Thus, if the context component 702 had knowledge that the user was not camping, and that it was near dinnertime, the utterance would be interpreted as “steak”. On the other hand, if the context component 702 knew (e.g., via GPS system input) that the user recently arrived at a camping ground within a national park; it might more heavily weight the utterance as “stake”.

In view of the foregoing, it is readily apparent that utilization of the context component 702 to consider and analyze extrinsic information can substantially facilitate determining meaning of sets of inputs.

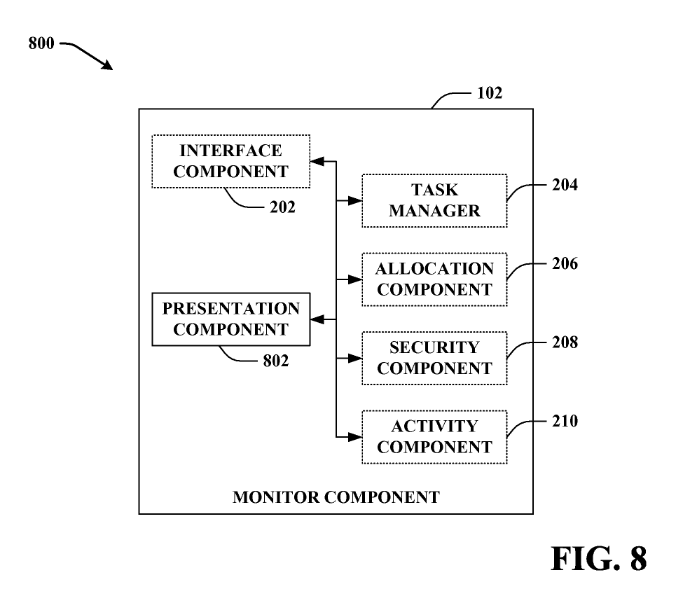

FIG. 8 provides further illustration of a system 800 that effectuates and/or facilitates control and/or regulation over an individual's physical or mental activities in accordance with an aspect of the claimed subject matter. As illustrated, system 800 can include presentation component 802 that can provide various types of user interface to facilitate interaction between a user and any component coupled to monitor component 102. As illustrated, presentation component 802 and/or other view components is incorporated within monitor component 102. However, as will be appreciated presentation component 802 can be utilized in conjunction with monitor component 102 as a standalone unit. Presentation component 802 can provide one or more graphical user interface, command line interface, and the like. For example, a graphical user interface can be rendered that provides the user with a region or means to load, import, read, etc., data, and can include a region to present the results of such. These regions can comprise known text and/or graphic regions comprising dialog boxes, static controls, drop-down menus, list boxes, pop-up menus, edit controls, combo boxes, radio buttons, check boxes, push buttons, and graphic boxes. In addition, utilities to facilitate the presentation such as vertical and/or horizontal scrollbars for navigation and toolbar buttons to determine whether a region will be viewable can be employed. For example, the user can interact with one or more of the components coupled and/or incorporated into monitor component 102.

Users can also interact with regions to select and provide information via various devices such as a mouse, roller ball, keypad, keyboard, and/or voice activation, for example. Typically, mechanisms such as a push button or the enter key on the keyboard can be employed subsequent to entering the information in order to initiate, for example, a query. However, it is to be appreciated that the claimed subject matter is not so limited. For example, merely highlighting a checkbox can initiate information conveyance. In another example, a command line interface can be employed. For example, the command line interface can prompt (e.g., via text message on a display and/or an audio tone) the user for information via a text message. The user can then provide suitable information, such as alphanumeric input corresponding to an option provided in the interface prompt or an answer (e.g., verbal utterance) to a question posed in the prompt. It is to be appreciated that the command line interface can be employed in connection with a graphical user interface and/or application programming interface (API). In addition, the command line interface can be employed in connection with hardware (e.g., video cards) and/or displays (e.g., black-and-white, and EGA) with limited graphic support, and/or low bandwidth communication channels.

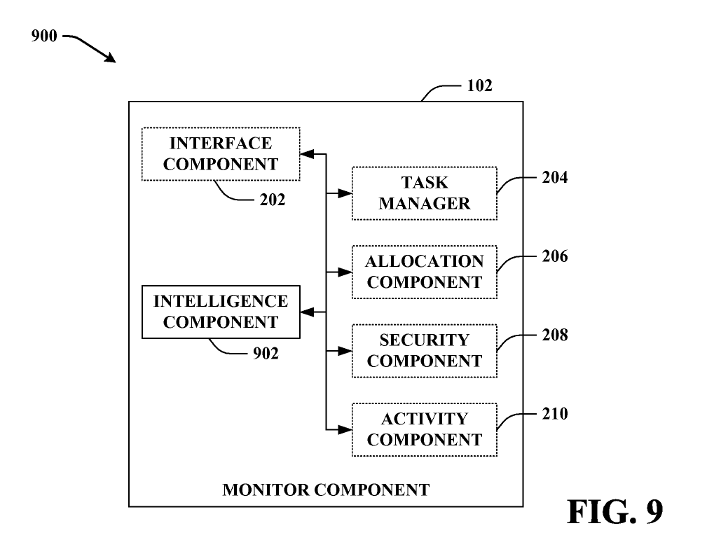

FIG. 9 depicts a system 900 that employs artificial intelligence to effectuate and/or facilitate control and/or regulation over an individual's physical or mental activities in accordance with an aspect of the subject matter as claimed. Accordingly, as illustrated, system 900 can include an intelligence component 902 that can employ a probabilistic based or statistical based approach, for example, in connection with making determinations or inferences. Inferences can be based in part upon explicit training of classifiers (not shown) before employing system 200, or implicit training based at least in part upon system feedback and/or users previous actions, commands, instructions, and the like during use of the system. Intelligence component 902 can employ any suitable scheme (e.g., neural networks, expert systems, Bayesian belief networks, support vector machines (SVMs), Hidden Markov Models (HMMs), fuzzy logic, data fusion, etc.) in accordance with implementing various automated aspects described herein. Intelligence component 902 can factor historical data, extrinsic data, context, data content, state of the user, and can compute cost of making an incorrect determination or inference versus benefit of making a correct determination or inference. Accordingly, a utility-based analysis can be employed with providing such information to other components or taking automated action. Ranking and confidence measures can also be calculated and employed in connection with such analysis.

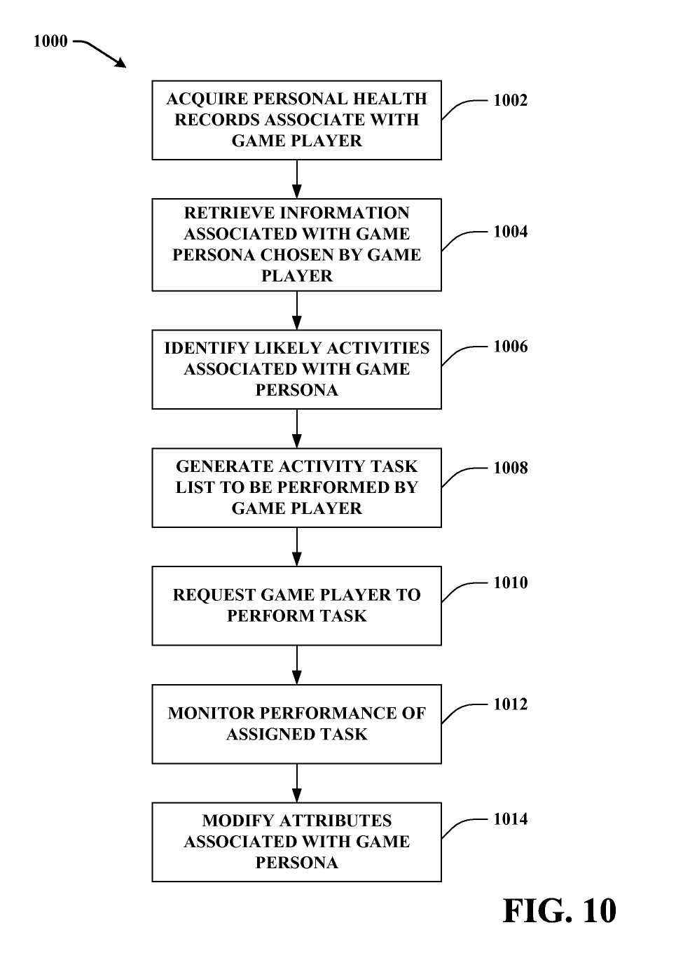

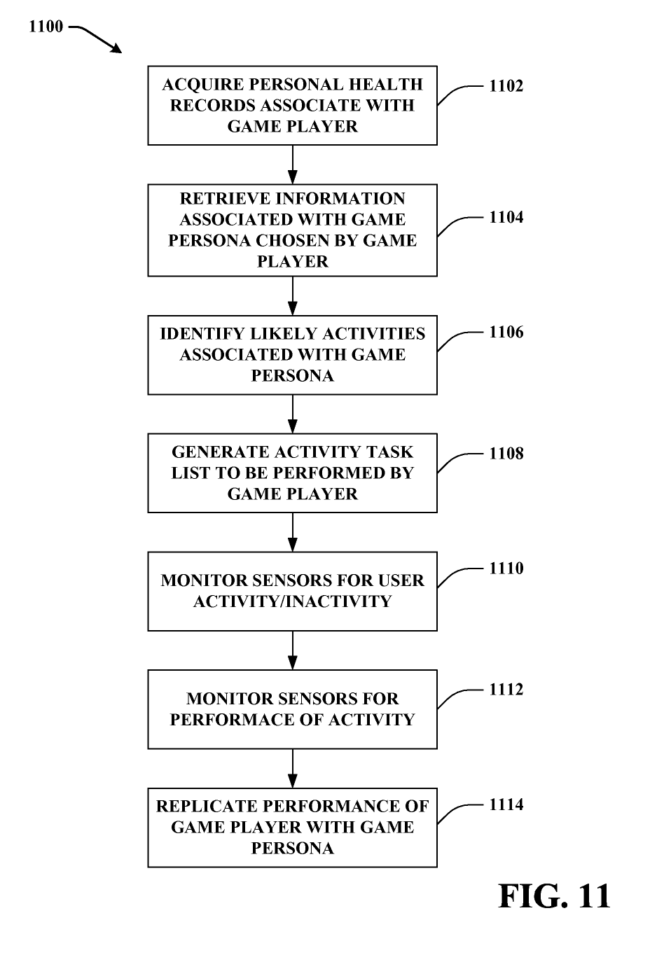

In view of the illustrative systems shown and described supra, methodologies that may be implemented in accordance with the disclosed subject matter will be better appreciated with reference to the flow charts of FIG. 10-11. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described hereinafter. Additionally, it should be further appreciated that the methodologies disclosed hereinafter and throughout this specification are capable of being stored on an article of manufacture to facilitate transporting and transferring such methodologies to computers.

The claimed subject matter can be described in the general context of computer-executable instructions, such as program modules, executed by one or more components. Generally, program modules can include routines, programs, objects, data structures, etc. that perform particular tasks or implement particular abstract data types. Typically the functionality of the program modules may be combined and/or distributed as desired in various aspects.

FIG. 10 illustrates a machine implemented method 1000 that encourages individuals to modify their behavior and perform an activity. Method 1000 can commence at 1002 where personal health records associated with a game player can be acquired. It should be noted that access to personal health records can require a great deal of authentication and utilization of cryptographic keys and biometric details in order to verify the authenticity of the parties attempting the access personal health records. At 1004 information related to a game persona associated with or chosen by a game player can be obtained. At 1006 activities associated with the game persona can be identified. At 1008 a set of tasks based at least in part on the activities associated with the game persona and/or the personal health records associated with the game player can be generated. The set of tasks generated at 1008 can be individualized or customized to be suitable for the game player. Such customization can include selecting tasks according to characteristics such as fitness levels, age, sex, preferences for specific activities, etc. At 1010 the game player can be requested to perform a task from the set of tasks. At 1012 performance of the assigned task can be monitored and at 1014 attributes associated with the game persona can be modified based on an objective analysis of the game player's performance of the requested task. For example, at 1014 where the objective assessment of the game player's performance is that it is, or was, comparatively substandard the game persona abilities, skills, or attributes can be diminished in some manner. Conversely, where at 1014 the objective assessment of the game player's performance is satisfactory or good, then the game persona's skills, abilities, and/or attributes can be augmented in some manner.

FIG. 11 provides illustration of a machine implemented methodology 1100 that regulated behavior in accordance with an aspect of the claimed subject matter. Method 1100 can commence at 1102 where personal health records associated with the game player can be acquired. At 1104 information associated with a game persona chosen by the game player can be retrieved. At 1106 likely activities associated with the game persona can be identified (e.g., likely game activities undertaken by game persona can be dependent on the game itself, so action games can entail much more physical activity and/or manual dexterity than do more cogitative games which would require more mental acuity). At 1108 a list of activity tasks to be performed or undertaken by the game play can be generated. At 1110 sensors associated (e.g., attached to or in the vicinity of) with the game player can be utilized to determine whether the user is in an active or inactive state (e.g., this determination can be indicative of whether or not the game player is in a rested state or whether or not he or she has just completed some unrelated non-game related physical activity). At 1112 sensors associated with the game player can be employed to determine whether or not the game player is undertaking, or has undertaken the activity identified and prescribed. At 1114 where sensors at 1112 detect that the game player is currently undertaking, or has undertaken, the prescribed activity, the game player's performance can be projected onto the performance of the game persona wherein the game persona can mimic the activity undertaken by the game player and emulate the intensity or lack thereof of the game player in performing the assigned and indicated task.

The claimed subject matter can be implemented via object oriented programming techniques. For example, each component of the system can be an object in a software routine or a component within an object. Object oriented programming shifts the emphasis of software development away from function decomposition and towards the recognition of units of software called “objects” which encapsulate both data and functions. Object Oriented Programming (OOP) objects are software entities comprising data structures and operations on data. Together, these elements enable objects to model virtually any real-world entity in terms of its characteristics, represented by its data elements, and its behavior represented by its data manipulation functions. In this way, objects can model concrete things like people and computers, and they can model abstract concepts like numbers or geometrical concepts.

As used in this application, the terms “component” and “system” are intended to refer to a computer-related entity, either hardware, a combination of hardware and software, or software in execution. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Artificial intelligence based systems (e.g., explicitly and/or implicitly trained classifiers) can be employed in connection with performing inference and/or probabilistic determinations and/or statistical-based determinations as in accordance with one or more aspects of the claimed subject matter as described hereinafter. As used herein, the term “inference,” “infer” or variations in form thereof refers generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification schemes and/or systems (e.g., support vector machines, neural networks, expert systems, Bayesian belief networks, fuzzy logic, data fusion engines . . . ) can be employed in connection with performing automatic and/or inferred action in connection with the claimed subject matter.

Furthermore, all or portions of the claimed subject matter may be implemented as a system, method, apparatus, or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter.

Some portions of the detailed description have been presented in terms of algorithms and/or symbolic representations of operations on data bits within a computer memory. These algorithmic descriptions and/or representations are the means employed by those cognizant in the art to most effectively convey the substance of their work to others equally skilled. An algorithm is here, generally, conceived to be a self-consistent sequence of acts leading to a desired result. The acts are those requiring physical manipulations of physical quantities. Typically, though not necessarily, these quantities take the form of electrical and/or magnetic signals capable of being stored, transferred, combined, compared, and/or otherwise manipulated.

It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the foregoing discussion, it is appreciated that throughout the disclosed subject matter, discussions utilizing terms such as processing, computing, calculating, determining, and/or displaying, and the like, refer to the action and processes of computer systems, and/or similar consumer and/or industrial electronic devices and/or machines, that manipulate and/or transform data represented as physical (electrical and/or electronic) quantities within the computer's and/or machine's registers and memories into other data similarly represented as physical quantities within the machine and/or computer system memories or registers or other such information storage, transmission and/or display devices.

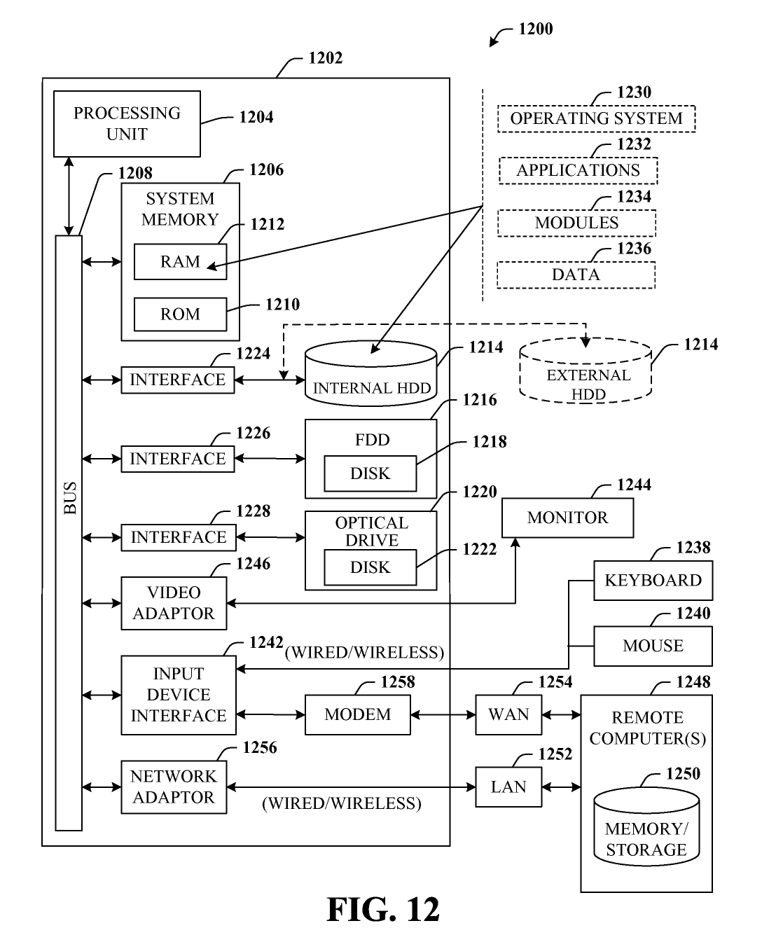

Referring now to FIG. 12, there is illustrated a block diagram of a computer operable to execute the disclosed system. In order to provide additional context for various aspects thereof, FIG. 12 and the following discussion are intended to provide a brief, general description of a suitable computing environment 1200 in which the various aspects of the claimed subject matter can be implemented. While the description above is in the general context of computer-executable instructions that may run on one or more computers, those skilled in the art will recognize that the subject matter as claimed also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the claimed subject matter may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and non-volatile media, removable and non-removable media. By way of example, and not limitation, computer-readable media can comprise computer storage media and communication media. Computer storage media includes both volatile and non-volatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital video disk (DVD) or other optical disk storage, magnetic cassettes, magnetic tape, magnetic disk storage or other magnetic storage devices, or any other medium which can be used to store the desired information and which can be accessed by the computer.

With reference again to FIG. 12, the illustrative environment 1200 for implementing various aspects includes a computer 1202, the computer 1202 including a processing unit 1204, a system memory 1206 and a system bus 1208. The system bus 1208 couples system components including, but not limited to, the system memory 1206 to the processing unit 1204. The processing unit 1204 can be any of various commercially available processors. Dual microprocessors and other multi-processor architectures may also be employed as the processing unit 1204.

The system bus 1208 can be any of several types of bus structure that may further interconnect to a memory bus (with or without a memory controller), a peripheral bus, and a local bus using any of a variety of commercially available bus architectures. The system memory 1206 includes read-only memory (ROM) 1210 and random access memory (RAM) 1212. A basic input/output system (BIOS) is stored in a non-volatile memory 1210 such as ROM, EPROM, EEPROM, which BIOS contains the basic routines that help to transfer information between elements within the computer 1202, such as during start-up. The RAM 1212 can also include a high-speed RAM such as static RAM for caching data.

The computer 1202 further includes an internal hard disk drive (HDD) 1214 (e.g., EIDE, SATA), which internal hard disk drive 1214 may also be configured for external use in a suitable chassis (not shown), a magnetic floppy disk drive (FDD) 1216, (e.g., to read from or write to a removable diskette 1218) and an optical disk drive 1220, (e.g., reading a CD-ROM disk 1222 or, to read from or write to other high capacity optical media such as the DVD). The hard disk drive 1214, magnetic disk drive 1216 and optical disk drive 1220 can be connected to the system bus 1208 by a hard disk drive interface 1224, a magnetic disk drive interface 1226 and an optical drive interface 1228, respectively. The interface 1224 for external drive implementations includes at least one or both of Universal Serial Bus (USB) and IEEE 1094 interface technologies. Other external drive connection technologies are within contemplation of the claimed subject matter.

The drives and their associated computer-readable media provide nonvolatile storage of data, data structures, computer-executable instructions, and so forth. For the computer 1202, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the illustrative operating environment, and further, that any such media may contain computer-executable instructions for performing the methods of the disclosed and claimed subject matter.

A number of program modules can be stored in the drives and RAM 1212, including an operating system 1230, one or more application programs 1232, other program modules 1234 and program data 1236. All or portions of the operating system, applications, modules, and/or data can also be cached in the RAM 1212. It is to be appreciated that the claimed subject matter can be implemented with various commercially available operating systems or combinations of operating systems.

A user can enter commands and information into the computer 1202 through one or more wired/wireless input devices, e.g., a keyboard 1238 and a pointing device, such as a mouse 1240. Other input devices (not shown) may include a microphone, an IR remote control, a joystick, a game pad, a stylus pen, touch screen, or the like. These and other input devices are often connected to the processing unit 1204 through an input device interface 1242 that is coupled to the system bus 1208, but can be connected by other interfaces, such as a parallel port, an IEEE 1094 serial port, a game port, a USB port, an IR interface, etc.

A monitor 1244 or other type of display device is also connected to the system bus 1208 via an interface, such as a video adapter 1246. In addition to the monitor 1244, a computer typically includes other peripheral output devices (not shown), such as speakers, printers, etc.

The computer 1202 may operate in a networked environment using logical connections via wired and/or wireless communications to one or more remote computers, such as a remote computer(s) 1248. The remote computer(s) 1248 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1250 is illustrated. The logical connections depicted include wired/wireless connectivity to a local area network (LAN) 1252 and/or larger networks, e.g., a wide area network (WAN) 1254. Such LAN and WAN networking environments are commonplace in offices and companies, and facilitate enterprise-wide computer networks, such as intranets, all of which may connect to a global communications network, e.g., the Internet.

When used in a LAN networking environment, the computer 1202 is connected to the local network 1252 through a wired and/or wireless communication network interface or adapter 1256. The adaptor 1256 may facilitate wired or wireless communication to the LAN 1252, which may also include a wireless access point disposed thereon for communicating with the wireless adaptor 1256.

When used in a WAN networking environment, the computer 1202 can include a modem 1258, or is connected to a communications server on the WAN 1254, or has other means for establishing communications over the WAN 1254, such as by way of the Internet. The modem 1258, which can be internal or external and a wired or wireless device, is connected to the system bus 1208 via the serial port interface 1242. In a networked environment, program modules depicted relative to the computer 1202, or portions thereof, can be stored in the remote memory/storage device 1250. It will be appreciated that the network connections shown are illustrative and other means of establishing a communications link between the computers can be used.

The computer 1202 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11x (a, b, g, etc.) to provide secure, reliable, fast wireless connectivity. A Wi-Fi network can be used to connect computers to each other, to the Internet, and to wired networks (which use IEEE 802.3 or Ethernet).

Wi-Fi networks can operate in the unlicensed 2.4 and 5 GHz radio bands. IEEE 802.11 applies to generally to wireless LANs and provides 1 or 2 Mbps transmission in the 2.4 GHz band using either frequency hopping spread spectrum (FHSS) or direct sequence spread spectrum (DSSS). IEEE 802.11a is an extension to IEEE 802.11 that applies to wireless LANs and provides up to 54 Mbps in the 5 GHz band. IEEE 802.11a uses an orthogonal frequency division multiplexing (OFDM) encoding scheme rather than FHSS or DSSS. IEEE 802.11b (also referred to as 802.11 High Rate DSSS or Wi-Fi) is an extension to 802.11 that applies to wireless LANs and provides 11 Mbps transmission (with a fallback to 5.5, 2 and 1 Mbps) in the 2.4 GHz band. IEEE 802.11g applies to wireless LANs and provides 20+ Mbps in the 2.4 GHz band. Products can contain more than one band (e.g., dual band), so the networks can provide real-world performance similar to the basic 10BaseT wired Ethernet networks used in many offices.

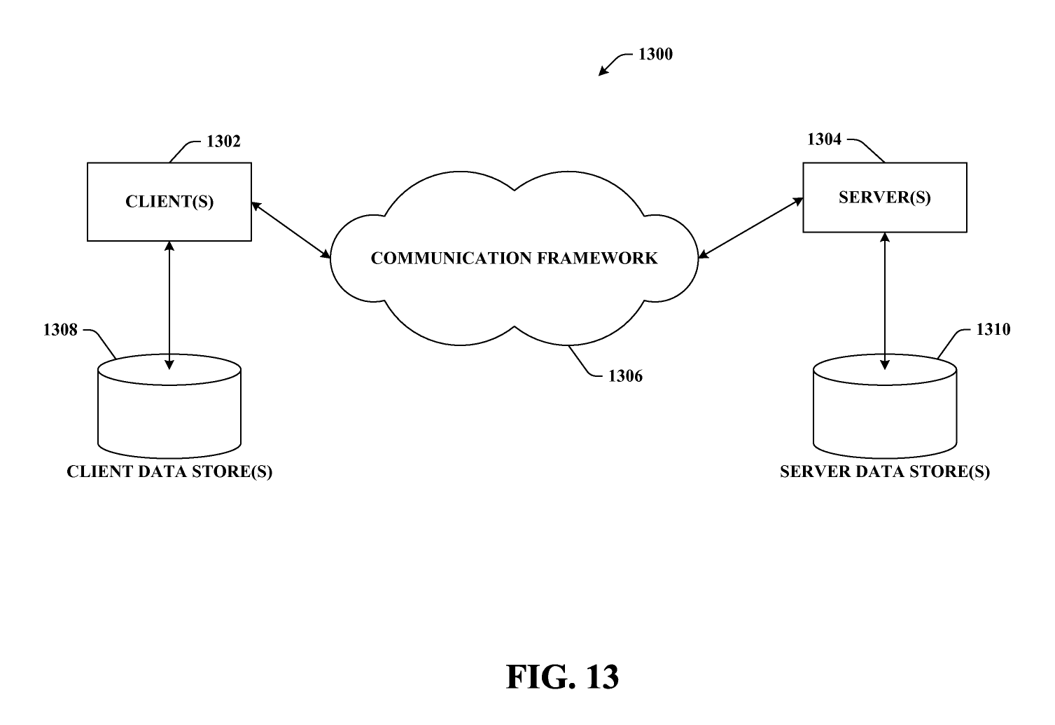

Referring now to FIG. 13, there is illustrated a schematic block diagram of an illustrative computing environment 1300 for processing the disclosed architecture in accordance with another aspect. The system 1300 includes one or more client(s) 1302. The client(s) 1302 can be hardware and/or software (e.g., threads, processes, computing devices). The client(s) 1302 can house cookie(s) and/or associated contextual information by employing the claimed subject matter, for example.

The system 1300 also includes one or more server(s) 1304. The server(s) 1304 can also be hardware and/or software (e.g., threads, processes, computing devices). The servers 1304 can house threads to perform transformations by employing the claimed subject matter, for example. One possible communication between a client 1302 and a server 1304 can be in the form of a data packet adapted to be transmitted between two or more computer processes. The data packet may include a cookie and/or associated contextual information, for example. The system 1300 includes a communication framework 1306 (e.g., a global communication network such as the Internet) that can be employed to facilitate communications between the client(s) 1302 and the server(s) 1304.

Communications can be facilitated via a wired (including optical fiber) and/or wireless technology. The client(s) 1302 are operatively connected to one or more client data store(s) 1308 that can be employed to store information local to the client(s) 1302 (e.g., cookie(s) and/or associated contextual information). Similarly, the server(s) 1304 are operatively connected to one or more server data store(s) 1310 that can be employed to store information local to the servers 1304.

What has been described above includes examples of the disclosed and claimed subject matter. It is, of course, not possible to describe every conceivable combination of components and/or methodologies, but one of ordinary skill in the art may recognize that many further combinations and permutations are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A machine implemented system that regulates behavior, comprising:
a first component that accesses via an interface personal health records associated with an individual;
a second component that probes an electronic game for at least one characteristic associated with a game character utilized by the individual to play the electronic game;
based at least in part on the personal health record associated with the individual or the at least one characteristic associated with the game character a third component identifies activities that the game character requests the individual to perform;
a fourth component that monitors a sensor associated with the individual for at least one of performance of the activities requested of the individual by the game character or inactivity of the individual; and
based at least in part on an intensity of the performance of the activities requested of the individual or information from the personal health records associated with the individual, a fifth component that at least one of augments an attribute associated with the game character or reduces the attribute associated with the game character.

2. The system of claim 1, the personal health records associated with the individual at least one of persisted on or associated with a manager component that performs a directed vertical search based on specifically tailored search queries that include health data items related to the individual's health.

3. The system of claim 1, the activities that the game character requests the individual to perform include at least one of a physical activity or a mental activity, the physical activity or the mental activity individualized based at least in part on the personal health records associated with the individual.

4. The system of claim 1, the activities that the game character requests the individual to perform utilized to at least one of accentuate or diminish the at least one characteristic associated with the game character.

5. The system of claim 1, the sensors associated with the individual include one or more of a heart rate monitor, a motion detector, a perspiration analyzer, a blood pressure monitor, a thermometer, or a global positioning system.

6. The system of claim 5, the sensors included in one or more of textiles, fabrics, clothing, or jewelry, the sensors communicate with the component via at least one of Near-Field Communications (NFC) or through electrical conductance associated with a human body.

7. The system of claim 1, the intensity of the performance of the activities requested of the individual derived from an analysis of the personal health records associated with the individual and input received from the sensor associated with the individual.

8. A method implemented on a machine that prescribes user behavior, comprising:
obtaining personal health records associated with the user;
retrieving game character attributes associated with a game;
identifying an activity undertaken by the game character during execution of the game;
requesting the user to perform a similar task to the activity undertaken by the game character during execution of the game;
monitoring the user performing the similar task to the activity undertaken by the game character during execution of the game; and
replicating an action of the user performing the similar task and projecting the action onto the game character where the game character performs the action during execution of the game.

9. The method of claim 8, the obtaining personal health records further comprising utilizing a cryptographic key to access authorized aspects of the personal health records associated with the user.

10. The method of claim 9, the cryptographic key employs one or more of an asymmetric public key scheme or a symmetric key scheme.

11. The method of claim 8, the monitoring the user performing the similar task further comprising utilizing sensors to determine a level of exertion being expended by the user in performing the similar task.

12. The method of claim 8, the personal health records associated with the user persisted on an engine that facilitates directed health specific searches, the directed health specific searches based at least in part on the persisted health records associated with the user or past search queries associated with the personal health records.

13. The method of claim 8, the similar task to the activity undertaken by the game character during execution of the game determined based at least on an age level of the user, a capability of the user, or a medical condition associated with the user.

14. The method of claim 8, the similar task to the activity undertaken by the game character includes at least one of a physical challenge or a mental challenge.

15. The method of claim 8, further comprising dynamically adjusting the similar task based at least in part on the monitoring the user performing the similar task.

16. A system that regulates behavior, comprising:
means for assigning to an individual an activity undertaken by a game persona during execution of a game;
means for monitoring performance of the activity assigned to the individual; and
means for projecting the performance of the activity by the individual to the game persona during execution of the game.

17. The system of claim 16, the means for assigning further comprising means for determining the activity based at least in part on a health record obtained from a means for vertically searching based on aspects associated with the health record.

18. The system of claim 16, the means for monitoring performance further comprising utilizing means for measuring blood pressure, means for locating a geographic location; and means for ascertaining pupil dilation.

19. The system of claim 16, the means for monitoring performance communicates with at least one of the means for measuring blood pressure, the means for ascertaining pupil dilation, a means for measuring heart rate, a means for determining body temperature, or a means for analyzing perspiration through at least one of Near-Field Communications or through electrical conductance imbued in the individual.

20. The system of claim 16, further comprising means for deriving an intensity level of the individual undergoing performance of the activity, the intensity level based at least in part on a personal health record associated with the individual.
